(19) United States
(12) Patent Application Publication
(10) Pub. No.: US 2006/0195085 A1
(43) Pub. Date: Aug. 31, 2006

(54) System and method for stabilizing spine

(75) Inventors: Harri Happonen, Tampere (FI); Vesa Vuorisalo, Tampere (FI)

Correspondence Address: BANNER & WITCOFF, LTD., 28 STATE STREET, 28th FLOOR, BOSTON, MA, 02109-9601, US

(73) Assignee: Inion Ltd., Tampere (FI)

(21) Appl. No.: 11/048,109

(22) Filed: Feb. 1, 2005

(51) Int. Cl. A61B 17/56; A61B 17/58

(52) U.S. Cl. 606/61

(57) ABSTRACT

A system and method for stabilizing the spine. The system comprises a fixation screw with a threaded section, and a fixation plate with a top and bottom surface. The fixation plate has a pre-drill hole arranged on the side of the top surface, and with the pre-drill hole, an actual fixation hole is made to the plate. The fixation hole is made when the plate is arranged at its future fixation point on the surface of bone tissue such that the fixation hole forms a first angle relative to the plate, whose size is selectable from several alternatives within certain limit values. The threaded section of the fixation hole is made during the same work phase as the threaded section in the bone tissue.

### BACKGROUND OF THE INVENTION

The invention relates to a system for stabilizing the spine, especially the cervical spine, which arrangement comprises a fixation plate and fixation screws with which the fixation plate is fastened to threaded holes made in the bone. Both the fixation plate and screws can be made of a biodegradable material that dissolves in the organ system.

The invention further relates to a method for stabilizing the spine, especially the cervical spine, in which method a stabilizing fixation plate is fastened through fixation screws to the part of the spine or cervical spine that is to be stabilized.

Various injuries may be caused to the spine due to degeneration, tumors, and fractures and dislocations caused by physical trauma. In some operations directed to the spine, fixation plates are used that are fastened to the bone tissue of the spine by means of fixation screws arranged through fixation holes in the fixation plate. Cervical spine operations are made either anteriorily or posteriority depending on the injury to be treated. A typical application of the fixation plate is the anterior ossification of cervical vertebrae. A damaged disc is removed from between the vertebrae and replaced for instance by a bone implant taken from the iliac to keep the intervertebral space intact. The vertebrae above and below the bone implant are fastened together with a fixation plate. The fixation plate is fastened to the vertebrae by screws. The fixation plate supports the vertebrae and prevents the bone implant from sliding away from between the vertebrae.

A method applicable to repairing damage to the spine and the fixation plates and screws used therein is disclosed in U.S. Pat. No. 5,601,553. A first fixation hole of the fixation plate described therein is perpendicular to a first plane of the top surface of the plate and, correspondingly, a second fixation hole is perpendicular to a second plane of the top surface of the plate. The first and second planes of the top surface intersect, whereby the axes of the fixation holes also intersect.

In this solution, as in other prior art arrangements, it may be problematic to arrange the fixation screws in such a manner that the fixation plate fastens appropriately to the bone tissue.

### BRIEF DESCRIPTION OF THE INVENTION

According to the invention presented above, a novel solution is obtained for the above-mentioned problem.

According to the present invention, the system for stabilizing the spine comprises a fixation screw with a threaded section, a fixation plate with a top and bottom surface, wherein the bottom surface is to be arranged against the bone tissue and the fixation plate has a pre-drill hole that is arranged on the side of said top surface and by means of which the actual fixation hole extending through the plate from the top surface to the bottom surface will be made, the fixation hole being arranged to be made when the plate is arranged at its future fixation point on the surface of the bone tissue in such a manner that the fixation hole forms a first angle in relation to the plate, the size of the first angle being selected from several alternatives within specific limit values, and the threaded section of the fixation plate being arranged to be made during the same work phase as the threaded section of the bone tissue, and the fixation screw being arranged to fit into the threaded section of the fixation plate and the threaded section of the bone tissue so as to lock the screw to the fixation plate.

The invention provides the advantage that the fixation screw can be arranged to such a position relative to the fixation plate that the fixation screw and thus also the fixation plate can be fixed to the bone tissue in the best possible manner taking into consideration medical and operational criteria.

An idea of an embodiment of the invention is that the pre-drill hole is a through hole that extends from the top surface of the plate to the bottom surface. An advantage is that, in a through hole, it is very easy to make a fixation hole whose center axis is coaxial with the center axis of the pre-drill hole.

An idea of an embodiment of the invention is that the pre-drill hole is a blind hole that extends to a distance from the bottom surface of the plate. An advantage is that the making of the fixation hole is then essentially as easy in any direction.

An idea of an embodiment of the invention is that the top surface of the fixation plate has a countersink and that the head of the fixation screw is designed with respect to the countersink in such a manner that it does not form an essential protuberance above the plane of the top surface of the fixation plate. This provides the advantage that the height of the system can be kept as small as possible, which means that it disturbs the neighboring tissue as little as possible.

An idea of an embodiment of the invention is that the thread of the threaded section of the fixation screw is arranged to be changeable so that the height of the screw ridge is greater in the section closer to the distal end than in the section further away from the distal end. This provides the advantage that a high ridge fastens optimally to the cancellous bone, whereas a low ridge fastens optimally to cortical bone.

The invention also relates to a method for stabilizing the spine, the method comprising: selecting a fixation plate having a top surface and a bottom surface and the top surface having a pre-drill hole arranged thereto, arranging the bottom surface of the fixation plate at its intended fastening point against the surface of the bone tissue to be stabilized, selecting the position of the fixation hole to be made through the pre-drill hole in relation to the fixation plate, the position being selectable among positions limited within specific limit values, making an actual fixation hole extending from the top surface of the plate to the bottom surface and, in the bone tissue, a screw hole that is coaxial and parallel with the fixation hole, threading both the fixation hole of the plate and the screw hole of the bone tissue to form a threaded section that continues substantially uniformly from the fixation hole to the screw hole in the bone tissue, tightening the fixation screw into the fixation hole and on through it to the screw hole in the bone tissue so that the fixation plate fastens to the bone tissue and, at the same time, the screw locks to the fixation plate.

### BRIEF DESCRIPTION OF THE FIGURES

The invention is described in greater detail in the attached drawings, in which

FIGS. 1a to 1c are schematic representations of a fixation plate belonging to the arrangement of the invention,

FIGS. 2a and 2b are schematic representations of a fixation screw belonging to the arrangement of the invention,

FIG. 3 is a schematic representation of an arrangement of the invention as seen from the end and with the fixation plate in cross-section,

FIGS. 4a to 4c are schematic representations of a second fixation plate belonging to the arrangement of the invention,

FIGS. 5a and 5b are schematic representations of a second fixation screw belonging to the arrangement of the invention,

FIG. 6 is a schematic representation of a second arrangement of the invention as seen from the end and with the fixation plate in cross-section, and

FIGS. 7a to 7c are schematic representations of a third fixation plate belonging to the arrangement of the invention.

In the figures, the invention is shown simplified for the sake of clarity. Similar parts are marked with the same reference numbers in the figures.

### DETAILED DESCRIPTION OF CERTAIN PREFERRED EMBODIMENTS

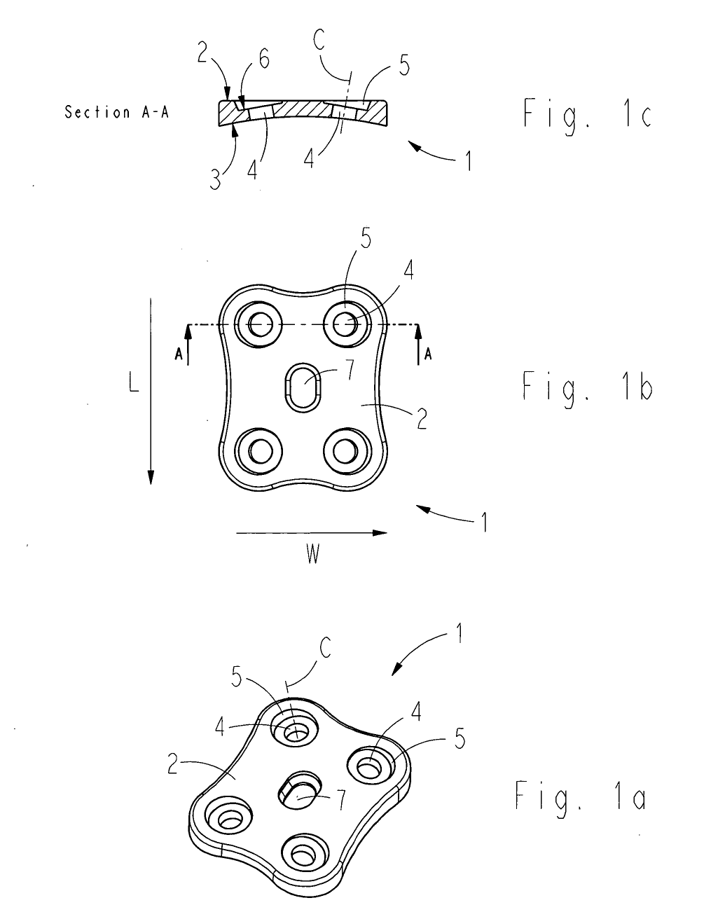

FIG. 1a is a schematic representation of a fixation plate belonging to the arrangement of the invention in perspective, FIG. 1b shows the same fixation plate from the direction of the top surface, and FIG. 1c shows it from the side in cross-section along section A-A. It should be noted that the fixation plate is in later on referred to as ‘plate’.

The plate 1 is preferably made of a biodegradable polymer material absorbing into the organ system that is prepared by polymerising or copolymerising for instance lactic acid, L-lactide, D-lactide, D,L-lactide, mesolactide, glycolic acid, glycolide or a cyclic ester copolymerised with lactide, or of any other corresponding material known per se to a person skilled in the art, which will not be discussed in this context in greater detail. Other suitable biodegradable polymers, copolymers and polymer mixtures are listed in the following publications, for instance:
“Encyclopedic Handbook of Biomaterials and Bioengineering, Part A,” Donald, L. Wise, Debra J. Trantolo, David E. Altobelli, Michael J. Yaszemski, Joseph D. Gresser, Edith R. Schwartz, 1992, by Marcel Dekker, Inc., pages 977 to 1007,
“Biodegradable fracture-fixation devices in maxillofacial surgery,” R. Suuronen, Int. J. Oral Maxillofac. Surg., 1993, 22: 50 to 57,
“Critical Concepts of Absorbable Internal Fixation,” William S. Pietrzak, Portland Bone Symposium, Portland, Oreg., Aug. 4 to 7, 1999,
“High-impact poly(L/D-lactide) for fracture fixation: in vitro degradation and animal pilot study,” Jan Tams, Cornelis A. P. Joziasse, Ruud R. M. Bos, Fred R. Rozema, Dirk W. Grijpma and Albert J. Pennings, Biomaterilas 1995, Vol. 16, No. 18, pages 1409 to 1415,
“A Review of Material Properties of Biodegradable and Bioresorbable Polymers and Devices for GTR and GBR Applications,” Dietmar Hutmacher, Markus B. Hürzeler, Henning Schliephake, The International Journal of Oral & Maxillofacial Implants, Volume 11, Number 5, 1996, pages 667 to 678, and
“Orthopaedic Application for PLA-Pga Biodegradable Polymers,” Kyriacos A. Athanasiou, Mauli Agrawal, Alan Barber, Stephen S. Burkkhart, The Journal of Arthroscopic and Related Surgery, Vol. 14, No. 7 (October), 1988: 726 to 737.

Further, it is obvious to a person skilled in the art that the material can be a composite that contains two or more materials or monomers, polymer chains, having the essential property of dissolving in the system. A composite can contain bio-glass, bio-ceramics, biologically active components, a pharmaceutical, such as antibiotic or growth factor, or the like.

Further, the material may comprise softeners, such as a pyrrolidone plasticizer. The pyrrolidones useful in the implants or methods of the present invention are any pyrrolidones known in the art of chemistry to have plasticizing properties without having tissue impairing effects or toxic effects. Such pyrrolidones include alkyl- or cycloalkyl-substituted pyrrolidones, such as N-methyl-2-pyrrolidone (NMP), 1-ethyl-2-pyrrolidone (NEP), 2-pyrrolidone (PB), and 1-cyclohexyl-2-pyrrolidone (CP), with NMP and NEP being preferred examples.

When examining the structure of the plate, it is detected that it has a top surface 2 and a bottom surface 3. The bottom surface 3 is intended to be arranged against the bone tissue to be stabilized and, correspondingly, the top surface 2 away from it. In the plates 1 shown in the figures, the top surface 2 has a substantially straight profile. The cross-section of the bottom surface 3 is, however, curved so that the bottom surface 3 curves with a straight line parallel to the longitudinal direction L as the curvature axis of the plate 1. The shape of the surfaces 2, 3 and other shapes of the plate 1 can naturally differ from the shapes shown in the figures.

A pre-drill hole 4 is formed on the top surface 2 of the plate and, in this case, it is a rotationally symmetrical and cylindrical through-hole extending from the top surface 2 to the bottom surface 3 and its imaginary longitudinal center axis is marked by reference mark C. The plate 1 has four pre-drill holes 4 in total, but their number may vary.

A countersink 5 is formed at the mouth of the pre-drill hole 4. The bottom plane 6 of the countersink 5 is substantially perpendicular to the center axis C; in other words, the center axis C is the diagonal of the spatial plane in the direction of the bottom plane 6.

The plate 1 also comprises a guide hole 7 arranged at the symmetrical center point of the plate 1. In the embodiment shown in the figures, the guide hole 7 is an oval through-hole extending through the plate 1, to which an instrument can be detachably attached to assist in the handling of the typically small plate 1 during its installation. Such instruments are known per se, so they are not discussed in more detail herein. It is also clear that the guide hole 7 can also be shaped and placed otherwise, as is the fact that the plate 1 can also be implemented without the guide hole 7.

The plate 1 is fastened in place relative to the bone tissue in such a manner that, first, it is arranged correctly relative to the bone tissue. This step may include shaping steps of the plate, in which the plate 1 is bent or its shape modified in some other manner known per se so as to fit it appropriately on the surface of the bone tissue. After this, through the pre-drill hole 4 but not necessarily in the direction of its center axis C, a hole is drilled in the bone tissue under the plate 1. The hole drilled in the bone tissue and the actual fixation hole drilled through the pre-drill hole 4 to the plate 1 are threaded with a threading tool in such a manner that a uniform thread is made to them. After this, a fixation screw is tightened into the threaded hole—which thus is first formed of the threaded section in the plate 1 and then of the threaded section in the bone tissue. The making of the fixation hole and its fastening to the bone tissue are described later in more detail.

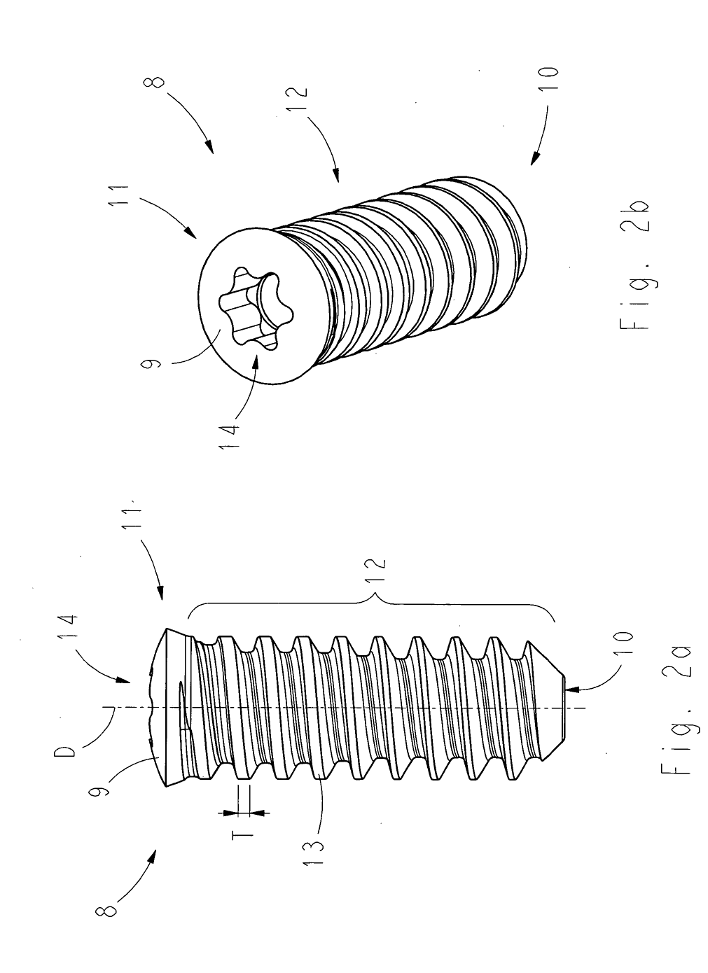

FIG. 2a is a schematic side view of a fixation screw belonging to the arrangement of the invention, and FIG. 2b is a perspective view from the direction of the proximal end. It should be noted that the fixation screw is later referred to as ‘screw’. The screw 8 can be made, for example, of the materials specified earlier as the manufacturing materials for the plate.

The screw 8 has a proximal end 9 and a distal end 10. A head 11 of the screw, the maximum diameter of which is at the same time the maximum diameter of the screw 8, is arranged at the proximal end 9. The ratio of the screw head 11 diameter to the outer diameter of the threaded section 12 is preferably 10:9-10:7.

A threaded section 12 is formed between the head 11 and the distal end 10. The threaded section of the screw shown in FIGS. 2a, 2b has one ridge 13 extending as a uniform thread from the head 11 to the distal end 10. The outer diameter of the threaded section 12 is constant with the exception of the convergent area of the top cone close to the distal end 10. The height of the ridge 13 is, however, smaller close to the proximal end 9 than to the distal end 10. In addition, the thickness T of the ridge 13 is greater close to the proximal end 9 than to the distal end 10 and in the section close to the proximal end 9.

The threaded section 12 may also have two or more ridges 13, in which case it is a multi-end threaded section. The ridge 13 can be discontinuous in that, at one point, it is completely or partially cut. In addition, the profile, height and thickness of the ridge 13 may be constant substantially throughout the length of the threaded section. The pitch of the threaded section 12 may also be different at different parts thereof.

The screw 8 has an imaginary longitudinal center axis D, around which the screw 8 rotates when the threaded section 12 is tightened to its counter-threads.

A counterpart 14 is arranged to the proximal end 9 for a tool required to tighten the screw. It should be noted that the tool is not shown in the figure. In its presented embodiment, the counterpart 14 resembles a Torx® driver, but it can naturally also be shaped otherwise.

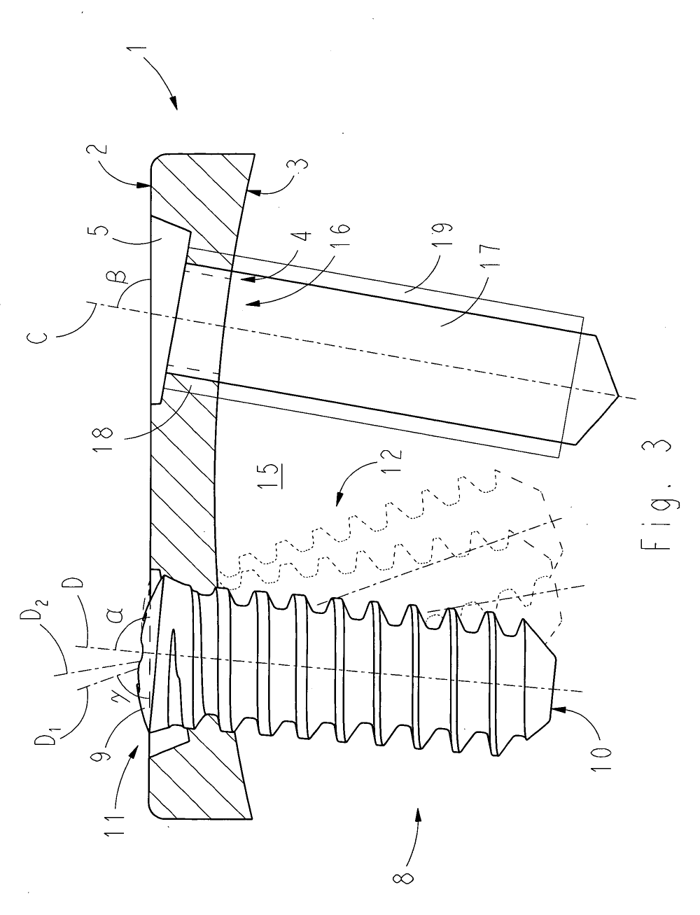

FIG. 3 is a schematic representation of an arrangement of the invention comprising the plate 1 shown in FIGS. 1a to 1c and the screws 8 shown in FIGS. 2a, 2b as seen from the end and with the plate 1 in cross-section.

Actual fixation holes 16 are made into the plate 1. The fixation holes 16 are made using the pre-drill holes 4—drawn by dashed lines to the rightmost fixation hole 16 only in FIG. 3—and the threaded sections 18 of the fixation holes are made to them.

A bone tissue screw hole 17 is made to the bone tissue 15 and a threaded section 19 of the bone tissue is made to it. The bone tissue screw hole 17 and the fixation hole 16 are parallel and coaxial with each other, and they are made by preferably drilling them at the same time. Drilling the holes at the same time ensures that the holes 16, 17 are parallel and coaxial.

The threaded section 18 of the fixation hole and the threaded section 19 of the bone tissue are made with the same tool and during the same step in such a manner that the threaded sections 18, 19 form a continuous threaded section from the plate 1 to the bone tissue 15. The manufacturing material of the plate 1 allows the making of the fixation hole 16 and its threaded section 18 with for instance any tools used in making bone holes. Bone drills and screw taps known per se can be used in making the fixation holes 16, 17 and their threaded sections.

The plate 1 can be fastened to the bone tissue 15 for instance as follows.

An appropriately shaped plate 1 is arranged to its fastening point relative to the bone tissue 15. The instrument attached detachably to the guide hole 7 can be used herein.

Next, a hole of the required size and depth is drilled in situ through the pre-drill hole 4 in the plate to the bone tissue 15. The hole forms the fixation hole 16 and the bone tissue screw hole 17. Not only the above-mentioned instrument, but also a drill guide can be used in this to guide the drill at the correct angle to the plate 1 and to also protect the surrounding tissue from the revolving drill bit.

After this, threads are made to the hole with a screw tap or some other threading tool. The result is a thread that is formed of the threaded section 18 of the fixation hole in the plate 1 and the threaded section 19 in the bone tissue. A drill guide can be used to protect the surrounding tissue from the revolving threading tool.

Another alternative is to use a combination of a drill and screw tap, i.e. a self-tapping drill. This has a drilling tip and a threading section. In other words, the hole and its threading is done at the same time with the same revolving movement of the self-tapping drill. One such self-tapping drill is disclosed in US patent application 2004/0092950 which is hereby incorporated by reference in its entirety for all purposes.

It is also possible to use a compressing drill or a screw tap that does not remove bone, but compresses it tightly around the drill hole. It is naturally also possible to use a compressing or partially compressing combination of a drill and a screw tap.

The pre-drill hole 4 facilitates the fastening of the plate 1 to the bone tissue 15, because it forms a clear starting point on the top surface 2 side of the plate, from which the making of the fixation hole 16 can be started.

The fixation holes 16 are arranged at a first angle relative to the top surface 2 of the plate 1; in other words, the longitudinal center axis C of the fixation hole 16 and the center axis D of the screw 8 arranged therein form a first angle relative to the top surface 2 of the plate 1. In the embodiment shown in FIGS. 2a, 2b, the first angles of the left and right fixation holes are unequal with respect to each other: in the case of the left fixation hole 16, the size of the first angle is a and, in the case of the right fixation hole 16, the size of the first angle is β.

The size of the first angle and, thus, the angle of the center axis C of the fixation hole 16 and the longitudinal center axis D of the screw 8 arranged therein relative to the plate 1 can be selected case by case in the most appropriate manner. In connection with the left fixation hole 16, alternative positions for the longitudinal center axis D of the screw 8 are shown by dashed lines and marked by reference markings D₁ and D₂. For instance, if for the criteria set by the fastening of the plate 1 and the stabilization of the bone tissue 15, it were best that the center axis of the left screw 8 was in the position shown by reference marking D₁, i.e. at angle γ relative to the plate 1, a fixation hole 16 would be made using the left pre-drill hole with the first angle of the center axis C being the above-mentioned γ.

When the screw 8 is tightened into the fixation hole 16 and on to the screw hole 17 of the bone tissue, it tightens to the threads 18, 19 therein in such a manner that a rigid constraint is formed between it and the plate to support the fixation, even though the screw 8 did loosen slightly in the bone. A rigid constraint means that a substantial micro-movement or angle movement relative to the plate 1 does not take place in the screw 8. Due to the low and thick ridge 13 close to the head 11 of the screw, the screw 8 locks very well to the thread in the plate 1.

The size of the first angle can be selected to be within specific limit values. The maximum value of the first angle can be for instance within the range of 70° to 90°, preferably 75° to 85°. The first angle can be divided into two orthogonal components that are its projections in the longitudinal direction L and lateral direction W of the plate. In other words, the center axis C, D can form an angle differing from right-angle both in the longitudinal direction L and lateral direction W of the fixation plate. Thus, the center axis C, D can be tilted both in the longitudinal direction L and lateral direction W of the plate to obtain the best possible angle for the fixation hole 16 and to the screw 8. It should be noted that the longitudinal direction L and lateral direction W of the plate 1 are shown in FIGS. 1b, 4b, and 7b. The screws 8 are preferably not arranged parallel into the bone tissue 15, because screws 8 oriented in different directions lock the plate 1 tighter to the bone tissue 15 and increase the strength required to pull out the screws 8.

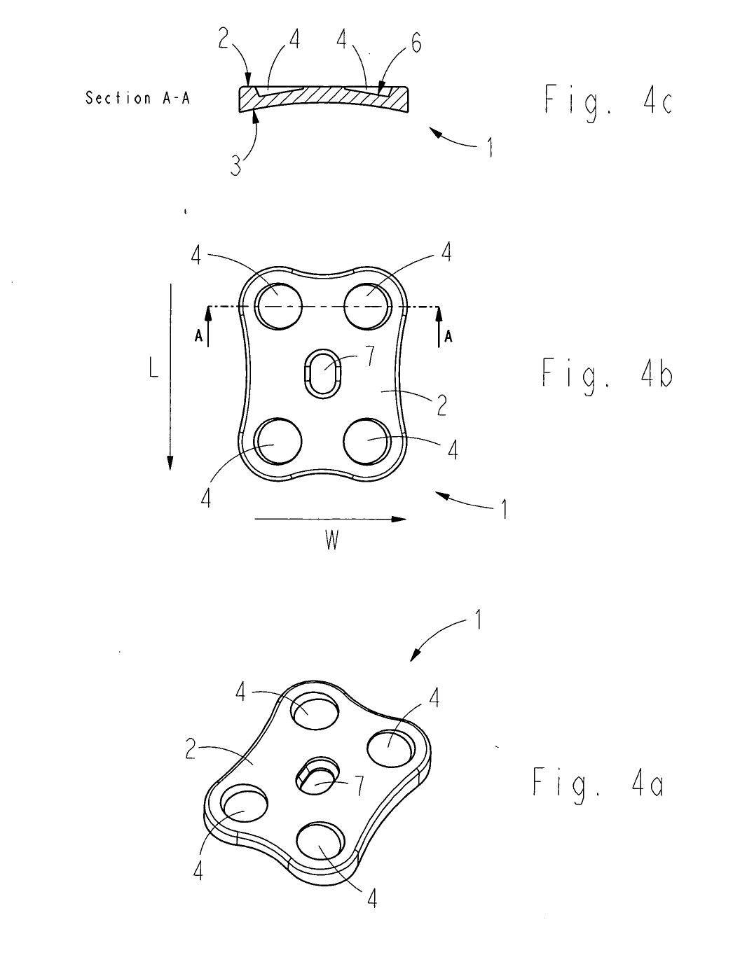

FIG. 4a is a schematic perspective view of a fixation plate belonging to a second arrangement of the invention, FIG. 4b shows the same fixation plate as seen from the top surface, and FIG. 4c from the side in cross-section along section A-A.

In this case, the plate 1 does not have a pre-drill hole through it, but the pre-drill hole 4 is a blind hole formed on the top surface 2 of the plate and extends to a distance from the bottom surface 3 of the plate. The basic form of the pre-drill hole 4 shown in FIGS. 4a to 4c is a cut circular cone with its center axis and bottom 6 arranged at an oblique angle relative to the top surface of the plate. Otherwise the plate 1 is mainly similar to the plate 1 shown in FIGS. 1a to 1c.

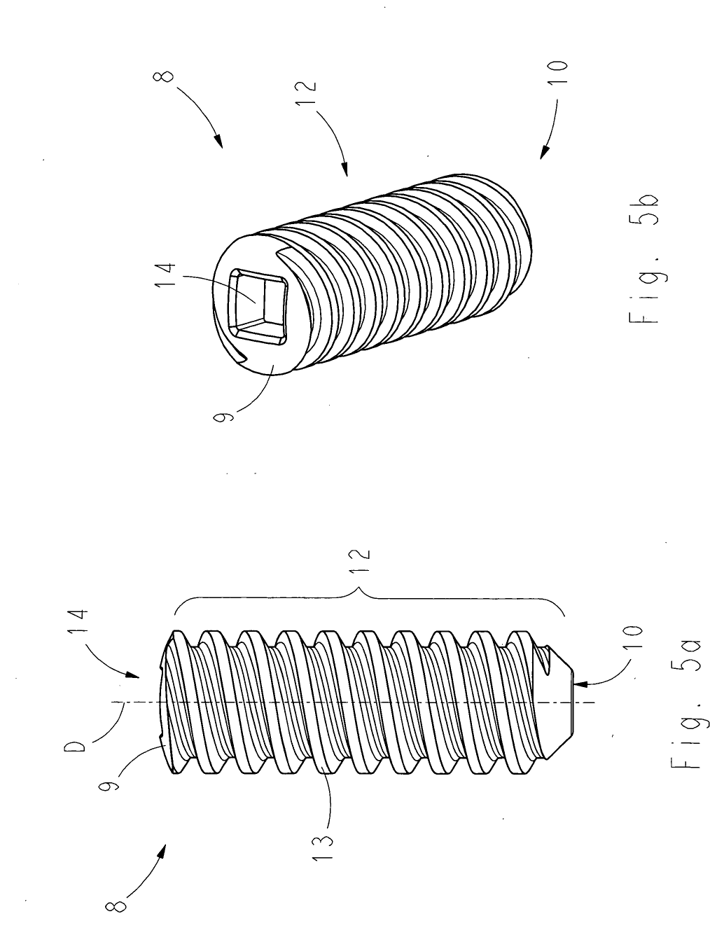

FIG. 5a is a schematic side view of a second fixation screw belonging to the arrangement of the invention and FIG. 5b is a perspective view thereof from the direction of the proximal end.

The screw 8 shown herein is a headless screw. The threaded section 12 of the screw 8 extends from the proximal end 9 to the distal end 10 and, thus, substantially along the entire length of the screw 8.

The threaded section 12 has a double-ended ridge 13, the height of which is constant substantially along the entire length of the threaded section 12. The thickness of the ridge 13 is also constant substantially along the entire length of the threaded section.

The screw 8 can be tightened into the fixation hole into such a depth that the proximal end 9 is level with the mouth of the fixation hole or even inside the fixation hole. The screw 8 then does not form a protrusion from the plate 1, which might have a disadvantageous impact on the tissues surrounding the arrangement.

A counterpart 14 with a square cross-section for a tool is arranged to the proximal end 9. The screw 8 can be tightened around its longitudinal center axis D with a tool arranged into the counterpart 14.

The screw 8 can be cannulated, i.e. a channel extending from the proximal end 9 to the distal end 10 and parallel to the center axis D can pierce it.

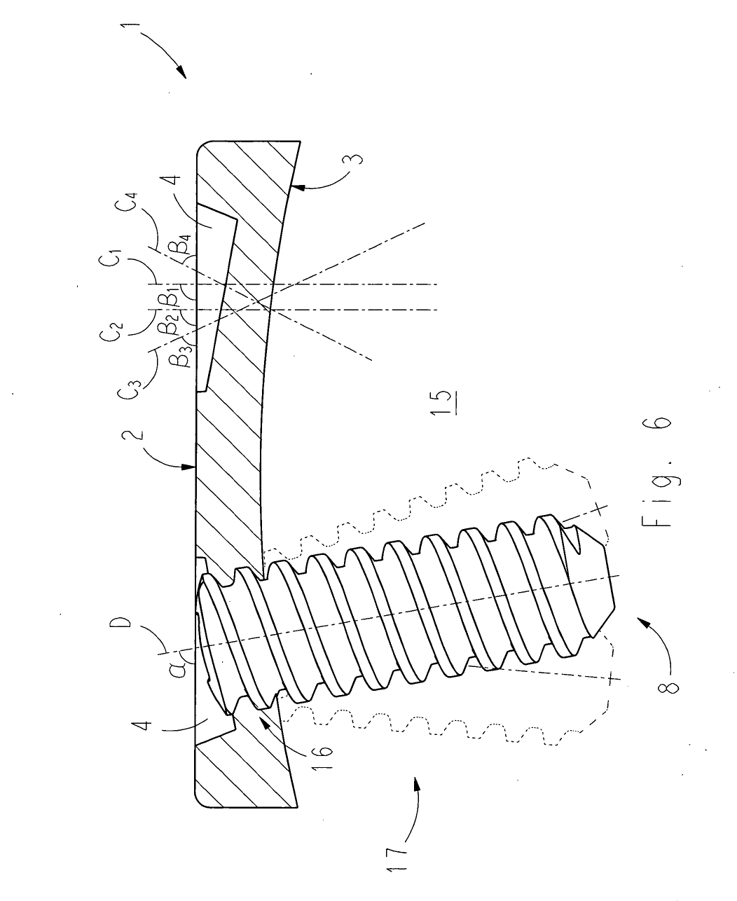

FIG. 6 is a schematic side view of a second arrangement of the invention that comprises the fixation plate shown in FIGS. 4a to 4c and the screws shown in FIGS. 5a, 5b, with the fixation plate in cross-section.

A fixation hole 16 and a bone tissue screw hole 17 and their uniform threaded sections are drilled in situ through the left pre-drill hole 4. A screw 8 is tightened into the threaded sections to fasten the plate 1 to the bone tissue 15. The longitudinal center axis C of the fixation hole 16 and thus also the longitudinal center axis D of the screw 8 are at a first angle relative to the top surface 2 of the plate. Herein, the size of the first angle is α. The fixation hole 16 could also have been made such that the first angle of the screw 8 tightened therein was other than α. Therefore, the figure shows two of such alternatives with dashed lines.

The left pre-drill hole 4 forms a countersink for the screw 8 on the top surface 2 of the plate. Due to the countersink, the proximal end 9 of the screw 8 tightened into the threaded section 18 of the fixation hole settles into the plate 1 so that is does not extend above the level of the top surface 2. Because of this, the total height of the system on the surface of the bone tissue 15 is very small.

In the situation shown in FIG. 6, a fixation hole has not yet been made through the right pre-drill hole 4. The figure shows a few alternatives for the position of the fixation hole to be made through the right pre-drill hole 4 as shown by means of the center axes C₁ to C₄ of the fixation holes.

When selecting the correct position of the fixation hole 16—and the screw 8 to be tightened therein—relative to the plate 1, it is possible to vary not only the angle of the fixation hole 16 relative to the plate 1—i.e. the first angle α, β₁ to β₄—but also the location where the mouth of the fixation hole 16 is made in the pre-drill hole 4. The mouth of the fixation hole 16 need not be arranged in the middle of the pre-drill hole 4, i.e. symmetrically to the pre-drill hole, but it can be arranged to the side in the longitudinal direction L and/or lateral direction W of the plate, i.e. asymmetrically to the pre-drill hole 4. The surgeon fastening the plate 1 has a great deal of choice in arranging each screw 8 to be arranged into the plate 1 individually in exactly the correct position relative to the plate 1 and bone tissue 15.

After the position and direction of the fixation hole to be made through the right pre-drill hole 4 and the bone tissue 15 screw hole to be made simultaneously in connection with it are selected, the fixation hole and bone tissue screw hole are made by drilling through the plate 1 to the bone tissue 15 a hole having the required diameter and length. Threads are made to this hole by using a threading tool known per se. The hole and threads can naturally also be made with a self-tapping drill. After making the threads, certain hole-finishing measures can be taken, after which a screw is tightened into the threads.

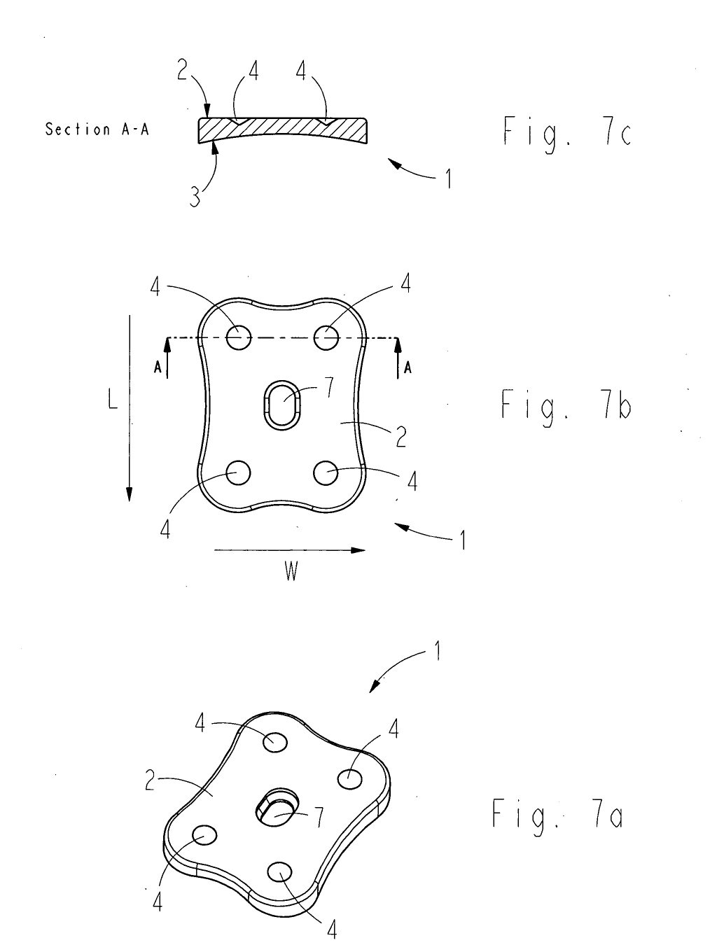

FIGS. 7a to 7c are schematic representations of a third fixation plate belonging to the arrangement of the invention. Like the fixation plates described above, this too can be made from a material that dissolves in the organ system.

The plate 1 comprises four pre-drill holes 4 arranged on the side of the top surface 2. The pre-drill hole 4 is now a blind hole in the shape of a circular cone and its tip forms the bottom of the pre-drill hole 4. The pre-drill hole 4 can also have some other shape than that shown in the figures: it can for instance be a tri- or multi-angular cone, a recess in the shape of a spherical surface, etc. The actual fixation hole and the required threaded sections are made as described in the preceding figures.

The drawings and the related description are only intended to illustrate the idea of the invention. The invention may vary in detail within the scope of the claims.

1. A system for stabilizing the spine, the system comprising:
a fixation screw with a threaded section,
a fixation plate with a top and bottom surface,
wherein the bottom surface is to be arranged against the bone tissue and the fixation plate has a pre-drill hole which is arranged on the side of said top surface and by means of which the actual fixation hole extending through the plate from the top surface to the bottom surface will be made,
the fixation hole being arranged to be made when the plate is arranged at its future fixation point on the surface of the bone tissue in such a manner that the fixation hole forms a first angle in relation to the plate, the size of the first angle being selected from several alternatives within specific limit values, and the threaded section of the fixation plate being arranged to be made during the same work phase as the threaded section of the bone tissue, and the fixation screw being arranged to fit into the threaded section of the fixation plate and the threaded section of the bone tissue so as to lock the screw to the fixation plate.

2. A system as claimed in claim 1, wherein the fixation plate and screw are made of a biodegradable material.

3. A system as claimed in claim 1, wherein the pre-drill hole extends from the top surface to the bottom surface of the plate.

4. A system as claimed in claim 1, wherein the pre-drill hole is a blind hole extending to a distance from the bottom surface of the fixation plate.

5. A system as claimed in claim 1, wherein a countersink is arranged at the top surface side end of the pre-drill hole.

6. A system as claimed in claim 1, wherein the fixation plate comprises a guide hole, to which a guide tool facilitating the handling of the fixation plate can be arranged.

7. A system as claimed in claim 6, wherein the guide hole is arranged at the symmetrical center point of the fixation plate.

8. A system as claimed in claim 1, wherein the pre-drill hole of the fixation hole forms a countersink of the fixation hole.

9. A system as claimed in claim 1, wherein the fixation hole is arranged asymmetrically to the pre-drill hole.

10. A system as claimed in claim 1, wherein the fixation screw comprises a proximal end with a counterpart for a tool arranged thereto.

11. A system as claimed in claim 10, wherein a screw head having a larger diameter than the maximum outer diameter of the threaded section is arranged at the proximal end.

12. A system as claimed in claim 11, wherein the ratio of the screw head diameter to the outer diameter of the threaded section is 10:9 to 10:7.

13. A system as claimed in claim 1, wherein the fixation plate has a countersink and the head of the fixation screw is designed relative to the countersink in such a manner that it fits below the top surface level of the plate.

14. A system as claimed in claim 1, wherein the fixation screw is a headless screw and the threaded section of the screw is arranged to extend to the proximal end.

15. A system as claimed in claim 1, wherein the thread of the threaded section of the fixation screw is arranged to change in such a manner that the height of the screw ridge is greater in the section close to the distal end than in the section further away from the distal end.

16. A system as claimed in claim 1, wherein the first angle forms an angle differing from right-angle both in the longitudinal and lateral direction of the fixation plate.

17. A method for stabilizing the spine, the method comprising:
selecting a fixation plate having a top surface and a bottom surface and the top surface having a pre-drill hole arranged therein,
arranging the bottom surface of the fixation plate at its intended fastening point against the surface of the bone tissue to be stabilized,
selecting the position of the fixation hole to be made through the pre-drill hole in relation to the fixation plate,
the position being selectable among positions limited within specific limit values,
making an actual fixation hole extending from the top surface of the plate to the bottom surface and, in the bone tissue, a screw hole that is coaxial and parallel with the fixation hole,
threading both the fixation hole of the plate and the screw hole of the bone tissue in such a manner that a threaded section is formed that continues substantially uniformly from the fixation hole to the screw hole in the bone tissue,
tightening the fixation screw into the fixation hole and on through it to the screw hole in the bone tissue so that the fixation plate fastens to the bone tissue and, at the same time, the screw locks into the fixation plate.

18. A method as claimed in claim 17, wherein the fixation plate and screws are made of a biodegradable material.
